(19) United States
(12) Patent Application Publication
(10) Pub. No.: US 2026/0345204 A1
(43) Pub. Date: Nov. 5, 2026

(54) WIRELESS FRACTURE REDUCTION ROBOT DEVICE AND OPERATION SYSTEM THEREFOR

(71) Applicant: AIRS INC., Daegu (KR)

(72) Inventor: Sanghyun JOUNG, Daegu (KR)

(21) Appl. No.: 18/992,875

(22) PCT Filed: Jun. 30, 2023
(86) PCT No.: PCT/KR2023/009197

§ 371 Date: Jan. 9, 2025

(30) Foreign Application Priority Data
Jul. 14, 2022 (KR) 10-2022-0086736

(51) Int. Cl. A61B 17/62; A61B 34/35; A61B 90/00; B25J 9/00; B25J 13/08

(52) U.S. Cl. CPC A61B 17/62; A61B 34/35; A61B 90/06; B25J 9/003; B25J 13/088; A61B 2090/061

(57) ABSTRACT

Provided is a wireless fracture reduction robot device including: a fracture reduction part having a plurality of ring frames adapted to surround a patient's fractured area and at least one or more struts located between the ring frames to adjust a distance between the ring frames; a wireless driving pack connected to any one of the ring frames to transmit power for adjusting the distance to the struts; sensing parts for measuring the positions of the struts; and a controller for producing length information of the struts, based on the positions of the struts measured from the sensing parts.

### TECHNICAL FIELD

The present invention relates to a wireless fracture reduction robot device and a system for operating the same, more specifically to a wireless fracture reduction robot device that is capable of allowing electric wires that cause interference with a surgeon's movement path during fracture reduction surgery to be removed therefrom, and to a wireless fracture reduction robot device-operating system that is capable of checking and controlling operations of the wireless fracture reduction robot device.

### BACKGROUND ART

Minimal invasive fracture reduction surgery is fracture reduction surgery that limits the size of an incision of a patient needed, and in the fracture reduction surgery, real-time x-ray imaging equipment such as a C-ARM device is used to allow a fractured bone to be put back in place. Next, intramedullary nails are inserted into the reduced fractured bone so that bone fragments are fixed thereto.

For such fracture reduction surgery, real-time x-ray images are acquired in a state where the fractured bone of the patient is located between an x-ray source of the C-ARM device and a two-dimensional sensor, and in this case, a surgeon performs the fracture reduction surgery, while monitoring the real-time x-ray images.

In this case, the fractured bone is located at a deep position inside the skin so that it is difficult to check the state of the fractured bone, the process for reducing the fractured bone, and the aligned state after the reduction of the fractured bone with the naked eye, and therefore, the fracture reduction surgery is generally performed using the real-time x-ray imaging equipment such as a C-ARM device.

To acquire real-time images, however, the real-time x-ray imaging equipment such as a C-ARM device has to irradiate x-rays constantly, which causes an amount of radiation exposure to the patient and the surgeon to be larger than that in other x-ray equipment for acquiring still images.

In this case, a surgeon who repeatedly performs the fracture reduction surgery has a big risk of such radiation exposure.

Further, since various muscles are connected to a bone, a large force is needed to reduce the fractured bone, and therefore, several surgeons generally perform the fracture reduction surgery together.

This requires a large number of surgeons for the fracture reduction surgery, which increases surgery costs.

Further, after the fractured bone has been reduced by the surgeons, it is not easy to accurately keep the reduced state until the reduced state is fixed through the insertion of the intramedullary nails. As a result, there is a possibility that the fracture reduction may result in an inaccurate fracture reduction.

As one of the conventional technologies, there is disclosed Korean Patent No. 10-1564717 entitled “Bone traction device and fracture reduction device having same”.

### DISCLOSURE

### Technical Problem

Accordingly, the present invention has been made in view of the above-mentioned problems occurring in the related art, and it is an object of the present invention to provide a wireless fracture reduction robot device and a system for operating the same that are capable of allowing lengths of struts for varying the positions of ring frames through which a patient's arm or leg passes to be measured to thus sense the shape of a fracture reduction part and allowing the shape of the fracture reduction part to be radio-controlled through a controller, thereby solving radiation exposure and labor difficulties and improving accuracy in fracture reduction surgery.

### Technical Solution

To accomplish the above-mentioned objects, according to one aspect of the present invention, there is provided a wireless fracture reduction robot device including: a fracture reduction part having a plurality of ring frames adapted to surround a patient's fractured area and at least one or more struts located between the ring frames to adjust a distance between the ring frames; a wireless driving pack connected to any one of the ring frames to transmit power for adjusting the distance to the struts; sensing parts for measuring the positions of the struts; and a controller for producing length information of the struts, based on the positions of the struts measured from the sensing parts.

Further, the wireless driving pack may include motor modules for transmitting the power to the struts, and each sensing part comprises an RPM sensor for detecting an RPM of the corresponding motor module to measure the position of the corresponding strut.

Furthermore, each strut may have a conductor located in a longitudinal direction thereof in such a way as to allow an electric current to flow therealong.

In this case, each sensing part may have a resistance sensor for detecting resistance from the size of the electric current of the conductor to measure the position of the corresponding strut.

Moreover, each sensing part may include: a winding machine for winding a wire connected to the corresponding strut thereon; a spring for rotating the winding machine to wind or unwind the wire on/from the winding machine; and a wire sensor for detecting the length of the wire unwound according to the rotation of the winding machine to measure the position of the corresponding strut.

Besides, each strut may have a reflector varied in a size of a reflection surface thereof in the longitudinal direction thereof.

In this case, each sensing part may include a light sensor for irradiating light to the reflector, receiving the light reflected from the reflector, detecting an amount of reflection of the received light, and thus measuring the position of the corresponding strut.

To accomplish the above-mentioned objects, according to another aspect of the present invention, there is provided a wireless fracture reduction robot device-operating system including: a wireless fracture reduction robot device including a fracture reduction part having a plurality of ring frames adapted to surround a patient's fractured area and at least one or more struts located between the ring frames to adjust a distance between the ring frames, sensing parts for measuring the positions of the struts, and a controller for producing length information of the struts, based on the positions of the struts measured from the sensing parts; and a user terminal interlinking with the wireless fracture reduction robot device in such a way as to receive the length information of the struts, while receiving control information for the adjustment of the lengths of the struts from a user to transmit the received control information to the wireless fracture reduction robot device.

### Advantageous Effects

As mentioned above, the wireless fracture reduction robot device and the system for operating the same according to the embodiments of the present invention are configured to allow the lengths of the struts for varying the positions of the ring frames through which the patient's arm or leg passes to be measured, thereby accurately sensing the shape of the fracture reduction part, without any direct check by the user.

Accordingly, the wireless fracture reduction robot device and the system for operating the same allow a state in which the fractured bone of the patient is reduced to be accurately kept to thus prevent an inaccurate fracture reduction result from occurring.

Further, the wireless fracture reduction robot device and the system for operating the same allow the fracture reduction part to be radio-controlled to the shape requested by the user through the controller.

### BRIEF DESCRIPTION OF DRAWINGS

FIG. 1 is a block diagram showing a wireless fracture reduction robot device-operating system according to an embodiment of the present invention.

FIG. 2 is a perspective view showing a wireless fracture reduction robot device according to an embodiment of the present invention.

FIG. 3 is a side view showing a state in which the wireless fracture reduction robot device of FIG. 2 is fixed to a human body.

FIG. 4 is a block diagram showing the control for the wireless fracture reduction robot device of FIG. 2.

FIG. 5 is a perspective view showing a state where a strut and a motor module of a wireless driving pack of FIG. 2 are connected to each other.

FIG. 6 is a front view of FIG. 5.

FIG. 7 is a sectional view showing a first variation of the sensing part located on the strut according to the present invention.

FIG. 8 is a sectional view showing a second variation of the sensing part located on the strut according to the present invention.

FIG. 9 is a sectional view showing a third variation of the sensing part located on the strut according to the present invention.

### BEST MODE FOR INVENTION

Hereinafter, specific exemplary embodiments of the present invention are illustrated in the drawings and described in detail in the detailed description. However, this does not limit the invention within specific embodiments and it should be understood that the invention covers all the modifications, equivalents, and replacements within the idea and technical scope of the invention.

In the description, the same reference numerals will be used to describe the same components. An expression referencing a singular value additionally refers to a corresponding expression of the plural number, unless explicitly limited otherwise by the context. In this application, terms, such as “comprise”, “include”, or ‘have”, are intended to designate those characteristics, numbers, steps, operations, elements, or parts which are described in the specification, or any combination of them that exist, and it should be understood that they do not preclude the possibility of the existence or possible addition of one or more additional characteristics, numbers, steps, operations, elements, or parts, or combinations thereof.

All terms used herein, including technical or scientific terms, unless otherwise defined, have the same meanings which are typically understood by those having ordinary skill in the art. The terms, such as ones defined in common dictionaries, should be interpreted as having the same meanings as terms in the context of pertinent technology, and should not be interpreted as having ideal or excessively formal meanings unless clearly defined in the specification.

If it is determined that the detailed explanation on the well-known technology related to the present invention makes the scope of the present invention not clear, the explanation will be avoided for the brevity of the description. Embodiments of the present invention will be provided to allow the present invention to be more perfectly explained to a person skilled in the art.

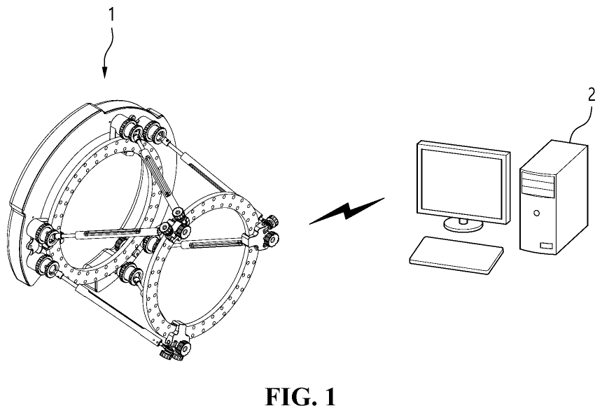

Hereinafter, an explanation of embodiments of the present invention will be given in detail with reference to FIGS. 1 to 9.

FIG. 1 is a block diagram showing a wireless fracture reduction robot device-operating system according to an embodiment of the present invention.

Referring to FIG. 1, a wireless fracture reduction robot device-operating system according to an embodiment of the present invention includes a wireless fracture reduction robot device 1 and a user terminal 2.

The wireless fracture reduction robot device 1 serves to reduce a patient's fractured bone and includes a fracture reduction part 100 having a plurality of ring frames 110 adapted to surround the patient's fractured bone area and at least one or more struts 120 located between the ring frames 110 to adjust a distance between the ring frames 110 and a controller 400 for measuring lengths of the struts 120 to produce length information.

In this case, the ring frames 110 are divided into a distal ring frame 111 and a proximal ring frame 112 according to positions thereof.

The wireless fracture reduction robot device 1 will be described in detail later.

The user terminal 2, which interlinks with the wireless fracture reduction robot device 1, receives the length information of the struts 120 and the relative positions/postures between the distal ring frame 111 and the proximal ring frame 112 from the wireless fracture reduction robot device 1, and further, the user terminal 2 receives control information for the adjustment of the lengths of the struts 120 from a user and thus transmits the received control information to the wireless fracture reduction robot device 1.

In this case, the user terminal 2 represents a PC, a mobile terminal, a tablet, and a Personal Digital Assistant (PDA), which are used by the user.

Further, the control information represents the information of the relative positions/postures between the distal ring frame 111 and the proximal ring frame 112 or the length information of the struts 120.

Furthermore, the user terminal 2 utilizes a pairing method using direct contact, such as near field communication (NFC), and a long distance wireless communication method using universal frequency 2.4 GHz ISM band and thus interlinks with the wireless fracture reduction robot device 1.

Besides, the user terminal 2 stores the length information received from the wireless fracture reduction robot device 1, infers the entire shape of the wireless fracture reduction robot device 1 according to the length information of the struts 120, and thus stores the inferred shape as shape information.

That is, the wireless fracture reduction robot device 1 transmits the information of the relative positions/postures between the distal ring frame 111 and the proximal ring frame 112 or the length information of the struts 120, which are varied according to the control information received from the user terminal 2, to the user terminal 2.

As a result, the user terminal 2 stores the data for a desirable fracture reduction with the received length information and provides big data related to fracture reductions according to patient characteristics to the user.

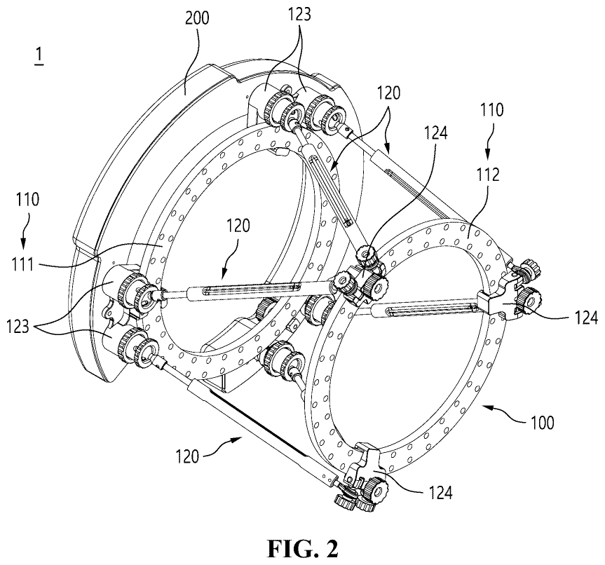

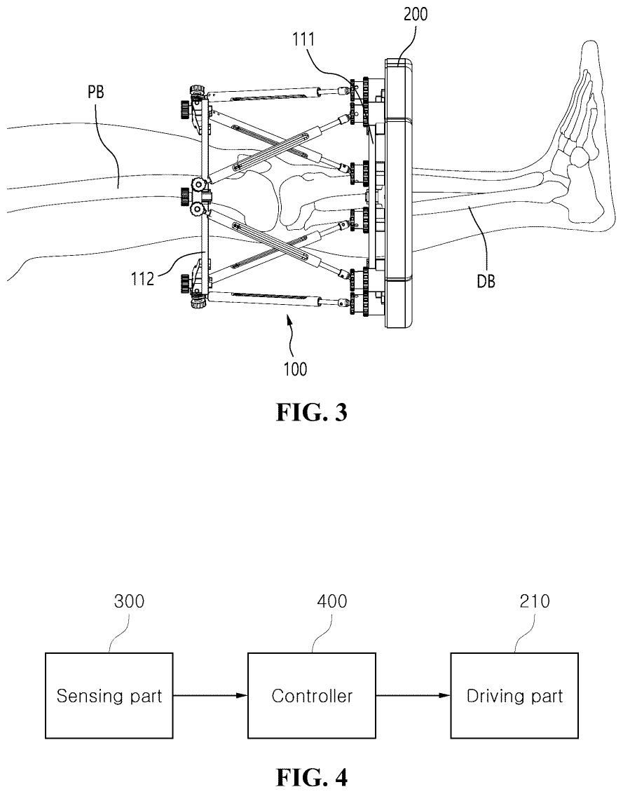

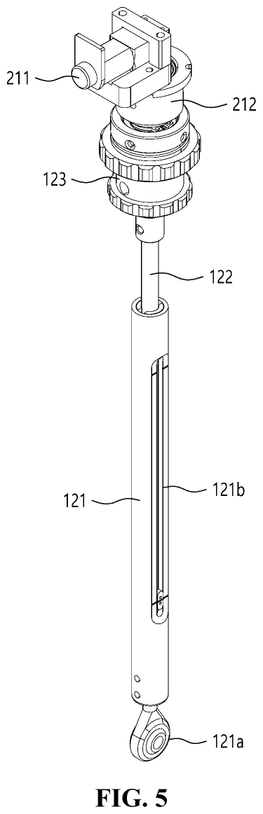

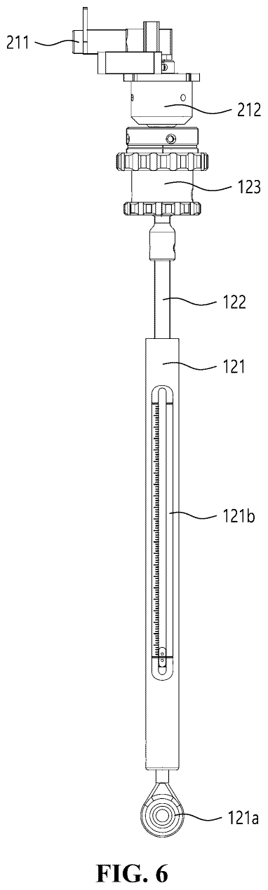

FIG. 2 is a perspective view showing a wireless fracture reduction robot device according to an embodiment of the present invention, FIG. 3 is a side view showing a state in which the wireless fracture reduction robot device of FIG. 2 is fixed to a human body, FIG. 4 is a block diagram showing the control for the wireless fracture reduction robot device of FIG. 2, FIG. 5 is a perspective view showing a state where a strut and a motor module of a wireless driving pack of FIG. 2 are connected to each other, and FIG. 6 is a front view of FIG. 5.

The wireless fracture reduction robot device 1 according to an embodiment of the present invention is configured to have a battery and a motor module 211 built therein to avoid electric wires from being exposed to the outside, and referring to FIGS. 2 to 4, the wireless fracture reduction robot device 1 includes the fracture reduction part 100, a wireless driving pack 200, and the controller 400.

The fracture reduction part 100 allows a patient's arm or leg to pass therethrough and is thus directly fixed to his or her fractured area. The fracture reduction part 100 serves to fix the fractured area thereto and pull it so that the fractured bone is reduced.

In this case, a fracture reduction is a procedure to restore fragments of a fractured bone or a dislocated bone to the correct alignment.

As shown in FIG. 2, the fracture reduction part 100 includes the ring frames 110 and the struts 120.

The ring frames 110 have the shapes of rings capable of allowing the patient's arm or leg to pass therethrough and are then fixed to the fractured area.

Further, the ring frames 110 are provided plurally so that they are spaced apart from each other by a given distance in such a way as to be fixed to the fractured area in a longitudinal direction of the fractured area.

Desirably, as shown in FIG. 3, the ring frames 110 are divided into the distal ring frame 111 and the proximal ring frame 112 that are fixed to a distal bone (DB) and a proximal bone (PB), respectively.

The struts 120 are connected between the distal ring frame 111 and the proximal ring frame 112.

One or more struts 120 are provided variable in length, like an actuator or cylinder, to allow an angle and a distance between the distal ring frame 111 and the proximal ring frame 112 to be adjusted.

In detail, referring to FIG. 5, each strut 120 includes a cylinder 121, a rod 122, a bottom connector 123, and a top connector 124.

The cylinder 121 is open on one end thereof and has a ring-shaped connection body 121a on the other end thereof in such a way as to be rotatably connected to one of the ring frames 110.

Further, the cylinder 121 has a sight gauge 121b located on the periphery thereof in a longitudinal direction thereof so that the position of the rod 122 is seen therethrough.

Further, referring to FIG. 6, measuring scales are formed in a longitudinal direction of the sight gauge 121b.

As a result, the lengths of the struts 120 are checked by the user through the sight gauges 121b, and further, the shape of the fracture reduction part 100 fixed to the fractured area is recognized according to the lengths of the struts 120.

The rod 122 moves to one end of the cylinder 121 inside the cylinder 121 in the longitudinal direction of the cylinder 121.

Further, one end of the rod 122 is connected to the ring frame 110 through the bottom connector 123 as will be discussed later and to the wireless driving pack 200 so that the rod 122 reciprocates inside the cylinder 121.

The bottom connector 123 has a clamp (not shown) along the periphery thereof so that it is brought into close contact with the periphery of the ring frame 110.

Further, the bottom connector 123 connects the rod 122 and the wireless driving pack 200 to each other and transfers a driving force to the rod 122 for allowing the rod 122 to reciprocate inside the cylinder 121.

The top connector 124 allows the connection body 121a to be rotatably connected thereto in such a way to apply six degrees of freedom allowing pitch, yaw, and roll rotations to the ring frame 110 according to the movements of the rod 122, that is, the changes in the length of the strut 120.

The changes in the lengths of the struts 120 enable the positions and postures of the distal ring frame 111 and the proximal ring frame 112 to be adjusted.

The wireless driving pack 200 includes driving parts 210 connected to the ring frame 110 in such a way as to transfer the power for the changes in the lengths of the struts 120 to the struts 120.

In detail, each driving part 210 has a motor module 211 built therein and a driving part coupler 212 protruding therefrom toward the strut 120 in such a way as to be connected to the bottom connector 123 to transfer the power of the motor module 211 to the strut 120.

The wireless driving pack 200 allows electric wires connected to the robot device, such as a communication wire, a power wire, and the like to be located therein, without any exposure to the outside.

Further, the wireless driving pack 200 has the shape of a ring through which the patient's arm or leg passes. In this case, the wireless driving pack 200 has an inner diameter greater than an outer diameter of the ring frame 110.

That is, the wireless driving pack 200 is bigger than the ring frames 110 to cause no interference with the movements of the ring frames 110 when the patient's arm or leg passes through the ring frames 110.

The wireless fracture reduction robot device 1 further includes sensing parts 300 adapted to measure the positions of the struts 120.

In this case, the sensing parts 300 may be located on the struts 120 of the fracture reduction part 100 or in the interior of the wireless driving pack 200 according to the user's request.

The sensing parts 300 will be explained in detail later.

The controller 400 is located inside the wireless driving pack 200 and produces the length information of the struts 120, based on the positions of the struts 120 measured through the sensing parts 300.

In this case, the controller 400 produces the length information of at least one or more struts 120 constituting the fracture reduction part 100.

Further, the controller 400 receives control information for adjustment in the lengths of the struts 120 to the wireless driving pack 200 from the user through the user terminal 2.

In this case, the controller 400 utilizes the pairing method using direct contact, such as near field communication (NFC), and the long distance wireless communication method using a universal frequency 2.4 GHz ISM band and thus communicates with the user terminal 2 and other terminals.

Further, the controller 400 transmits the length information of the struts 120 to the user terminal 2 so that the length information is checked by the user, and further, the controller 400 receives the control information for the adjustment in the lengths of the struts 120 from the user terminal 2 and transmits the received control information to the wireless driving pack 200.

As a result, the user adjusts the lengths of the struts 120, based on the received length information, so that the shape of the fracture reduction part 100 is controlled according to the patient's characteristics.

The controller 400 measures the lengths of the struts 120 constituting the fracture reduction part 100 and provides information inferring the entire shape of the fracture reduction part 100 according to the changes in the lengths of the struts 120.

Further, the user estimates desirable fracture reduction data from the length information produced from the controller 400 and builds big data related to the fracture reductions according to patient characteristics.

According to the embodiment of the present invention, each sensing part 300 includes an RPM (revolutions per minute) sensor (not shown).

The RPM sensor measures the RPM of the motor module 211 and thus produces senser information.

In this case, the sensor information refers to the position of the strut 120 measured through the RPM of the motor module 211 detected from the RPM sensor.

In detail, the RPM sensor is connected to a rotational shaft of the motor module 211 or a rotational shaft of the driving part coupler 212 and produces an electrical signal with one pulse per a revolution in a rotational direction of the rotational shaft to thus measure the RPM.

Further, the RPM sensor divides one revolution into 360 to subdivide the RPM and thus measure the RPM as rotational angles, which is set according to the user's request.

In this case, the controller 400 receives the sensor information produced from the rotations of the motor module 211 from the RPM sensor, determines the entire length of the strut 120, and produces the length information of the strut 120.

In detail, the controller 400 produces the length information of the strut 120, based on the sensor information detected from a resistance sensor 310, that is, based on an absolute value of a degree of movement of the rod 122 inside the cylinder 121 through the rotation of the motor module 211.

Otherwise, the sensing parts 300 may be located on the struts 120. Hereinafter, various variations of the sensing part 300 located on the strut 120 will be explained below.

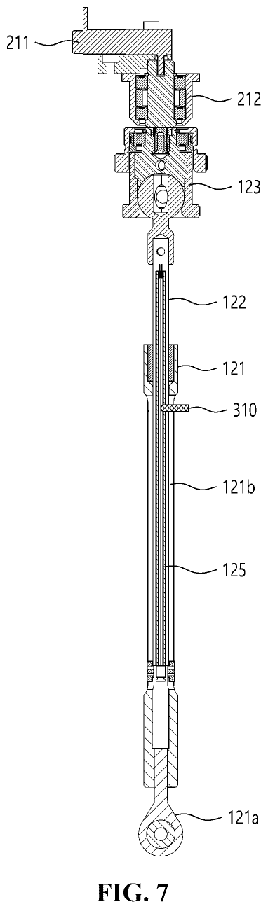

FIG. 7 is a sectional view showing a first variation of the sensing part located on the strut according to the present invention.

Referring to FIG. 7, the sensing part 300 of the wireless fracture reduction robot device 1 according to the embodiment of the present invention is replaced with a sensing part 300 according to a first variation of the present invention.

In this case, the strut 120 has a conductor 125 located in the longitudinal direction thereof in such a way as to allow an electric current to flow therealong.

The conductor 125 is located in the longitudinal direction of the rod 122 in such a way as to be exposed to the outside through the sight gauge 121b.

As the conductor 125 becomes distant from a portion to which the electric current is supplied, the conductor 125 interferes with the flow of the electric current because the number of collisions between electrons and atoms increases, so that a resistance increases in size.

The sensing part 300 according to the first variation of the present invention includes a resistance sensor 310.

The resistance sensor 310 measures resistance from the size of the electric current of the conductor 125 and thus produces sensor information.

In this case, the sensor information refers to the position of the strut 120 measured through a resistance size on the surface of the conductor 125 coming into contact with the resistance sensor 310.

In detail, the resistance sensor 310 is located close to the sight gauge 121b of the cylinder 121 in such a way as to face the sight gauge 121b and thus detects the electric current of the conductor 125.

The resistance sensor 310 detects resistance according to the variations of current intensity, while sensing the electric current flowing along the conductor 125, and thus produces the sensor information through a value of the detected resistance.

That is, the resistance sensor 310 is fixed in position and changed in positions coming into contact with the conductor 125 according to the movements of the rod 122, so that the resistance values detected are varied.

In this case, the controller 400 receives the sensor information as the resistance value varied according to the change in the position of the resistance sensor 310 through the movement of the rod 122, determines the entire length of the strut 120, and thus produces the length information of the strut 120.

In detail, the controller 400 allows the sensor information measured through the resistance sensor 310, that is, the resistance value measured according to the degree of movement of the rod 122 inside the cylinder 121 to match with the entire length of the strut 120 upon the movement of the rod 122 and thus produces the length information of the strut 120.

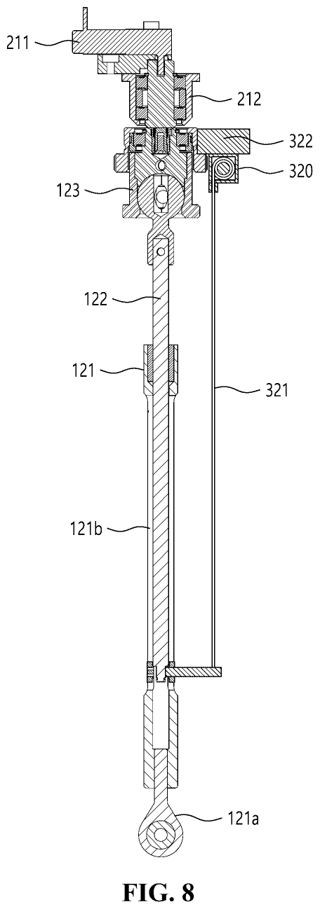

FIG. 8 is a sectional view showing a second variation of the sensing part located on the strut according to the present invention.

Referring to FIG. 8, the sensing part 300 of the wireless fracture reduction robot device 1 according to the embodiment of the present invention is replaced with a sensing part 300 according to a second variation of the present invention.

In this case, the sensing part 300 is located on the bottom connector 123 in such a way as to be open on one surface facing the other end of the rod 122 and thus wind or unwind a wire 321 according to the movements of the rod 122.

The sensing part 300 according to the second variation of the present invention includes a winding machine 320, the wire 321, a spring (not shown), and a wire sensor 322.

The winding machine 320 winds the wire 321 connected to the strut 120 thereon.

In this case, the wire 321 is connected to a wire fixing protrusion extending from the other end of the rod 122 toward the sight gauge 121b and thus wound or unwound on/from the winding machine 320 according to the movements of the rod 122.

The spring rotates the winding machine 320 to wind or unwind the wire 321 on/from the winding machine 320.

In detail, if tension is applied to the wire 321 connected to the rod 122 through the pulling of the rod 122, the spring rotates the winding machine 320 in a direction to which the tension is applied to allow the wire 321 to be unwound from the winding machine 320.

Further, the spring is provided with an elastic material wound thereon so that a force is applied to the center thereof, and accordingly, if the tension of the wire 321 becomes weak according to the insertion of the rod 122, the spring rotates the winding machine 320 in the opposite direction to the direction to which the tension is applied to allow the wire 321 to be wound on the winding machine 320, that is, to allow the wire 321 to be returned to its original state.

If the spring is wound or unwound, further, it provides elasticity to the wire 321 to prevent the wire 321 from being loose.

The wire sensor 322 detects the length of the wire 321 unwound according to the rotation of the winding machine 320 and produces the sensor information.

In detail, the wire sensor 322 is provided with a potentiometer (not shown) so that it generates a variable voltage or current according to the rotation of the winding machine 320, measures RPM according to the resistance value of the variable voltage or current to determine the length of the wire 321, and thus produces the sensor information.

Further, the wire sensor 322 recognizes the rotational direction of the winding machine 320, and thus, even if the same resistance values are measured, the wire sensor 322 determines whether the wire 321 is being wound or unwound.

In this case, the controller 400 receives the sensor information as the length information of the wire 321 according to the movement of the rod 122 from the wire sensor 322, determines the entire length of the strut 120, and thus produces the length information of the strut 120.

In detail, the controller 400 allows the sensor information measured through the wire sensor 322, that is, the length of the wire 321 measured according to the degree of movement of the rod 122 inside the cylinder 121 to match with the entire length of the strut 120 upon the movement of the rod 122 and thus produces the length information of the strut 120.

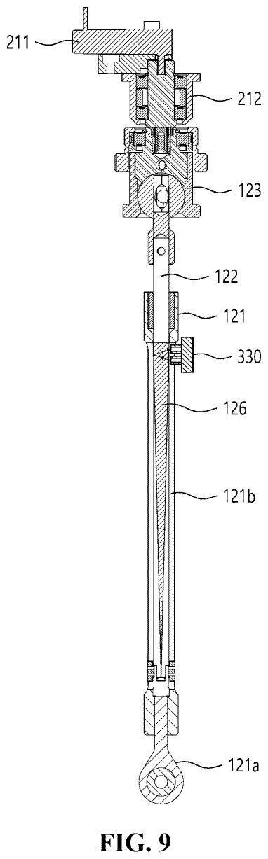

FIG. 9 is a sectional view showing a third variation of the sensing part located on the strut according to the present invention.

Referring to FIG. 9, the sensing part 300 of the wireless fracture reduction robot device 1 according to the embodiment of the present invention is replaced with a sensing part 300 according to a third variation of the present invention.

In this case, the strut 120 has a reflector 126 varied in a size of a reflection surface thereof in the longitudinal direction thereof.

The reflector 126 increases in size in the longitudinal direction of the rod 122 in such a way as to be exposed to the outside through the sight gauge 121b.

If light is incident on the reflector 126, the area of the reflected light from the reflector 126 increases in a longitudinal direction of the reflector 126.

The sensing part 300 according to the third variation of the present invention includes a light sensor 330.

The light sensor 330 irradiates light to the reflector 126, receives the light reflected from the reflector 126, measures an amount of reflection of the received light, and thus produces sensor information.

In detail, the light sensor 330 is located close to the sight gauge 121b of the cylinder 121 in such a way as to face the sight gauge 121b, irradiates light to the reflector 126, receives the light reflected from the reflector 126, measures the amount of reflection of the received light, and thus produces the sensor information.

In this case, the controller 400 receives the sensor information of the amount of reflection of the light varied according to the change in the position of the light sensor 330 through the movement of the rod 122, determines the entire length of the strut 120, and thus produces the length information of the strut 120.

In detail, the controller 400 allows the sensor information measured through the light sensor 330, that is, the amount of reflection of the light measured according to the degree of movement of the rod 122 inside the cylinder 121 to match with the entire length of the strut 120 upon the movement of the rod 122 and thus produces the length information of the strut 120.

Further, the respective components of the sensing part 300 according to the above-mentioned basic embodiment of the present invention and the respective components of the sensing parts 300 according to the first to third variations of the present invention may be replaced or combined with one another, while having no interference with one another, according to the user's request.

Further, the respective components of the sensing part 300 according to the above-mentioned basic embodiment of the present invention and the respective components of the sensing parts 300 according to the first to third variations of the present invention may have the same effectiveness.

As described above, the above-mentioned embodiment of the present invention may not be implemented only through a system and/or method, but it may be implemented through a system having the functions corresponding to the present invention and the components used for the system. Such implementation will be carried out by a person having ordinary skill in the art, based on the description of the present invention.

The foregoing description of the embodiments of the invention has been presented for the purpose of illustration; it is not intended to be exhaustive or to limit the invention to the precise forms disclosed. Persons skilled in the relevant art can appreciate that many modifications and variations are possible in light of the above teachings.

### EXPLANATIONS OF REFERENCE NUMERALS

1: Wireless fracture reduction robot device-operating system
100: Fracture reduction part
110: Ring frame
111: Distal ring frame
112: Proximal ring frame
120: Strut
121: Cylinder
121a: Connection body
121b: Sight gauge
122: Rod
123: Bottom connector
124: Top connector
125: Conductor
126: Reflector
200: Wireless driving pack
210: Driving part
211: Motor module
212: Driving part coupler
300: Sensor part
310: Resistance sensor
320: Winding machine
321: Wire
322: Wire sensor
330: Light sensor
DB: Distal bone
PB: Proximal bone

1. A wireless fracture reduction robot device comprising:
a fracture reduction part having a plurality of ring frames configured to surround a fractured area of a patient and one or more struts disposed between the plurality of ring frames to adjust a distance between the plurality of ring frames;
a wireless driving pack connected to any one of the plurality of ring frames to transmit power to the one or more struts for adjusting the distance between the plurality of ring frames;
one or more sensors for measuring a position of a corresponding strut; and
a controller for producing length information of the one or more struts, based on the positions of the one or more struts measured from the one or more sensors.

2. The wireless fracture reduction robot device of claim 1, wherein the wireless driving pack comprises one or more motor modules for transmitting the power to corresponding one or more struts, and
wherein each of the one or more sensor comprises an RPM sensor for detecting an RPM of a corresponding motor module to measure the position of the corresponding strut.

3. The wireless fracture reduction robot device of claim 1, wherein each of the one or more struts comprises a conductor disposed in a longitudinal direction of the strut to allow an electric current to flow along the conductor, and
wherein each of the one or more sensors comprises a resistance sensor for detecting resistance based on the electric current of the conductor to measure the position of the corresponding strut.

4. The wireless fracture reduction robot device of claim 1, wherein each of the one or more sensors comprises:
a winding machine for winding a wire connected to the corresponding strut thereon;
a spring for rotating the winding machine to wind or unwind the wire on or from the winding machine; and
a wire sensor for detecting a length of the wire unwound according to the rotation of the winding machine to measure the position of the corresponding strut.

5. The wireless fracture reduction robot device of claim 1, wherein each of the one or more struts comprises a reflector including a reflection surface having a varied size along a longitudinal direction of the strut, and
wherein each of the one or more sensors comprises a light sensor configured to irradiate light to the reflector, receive the light reflected from the reflector, detect an amount of reflection of the received light, and measure the position of the corresponding strut.

6. An operation system for a wireless fracture reduction robot device, the operation system comprising:
a wireless fracture reduction robot device comprising:
a fracture reduction part having a plurality of ring frames configured to surround a fractured area of a patient and one or more struts disposed between the plurality of ring frames to adjust a distance between the plurality of ring frames;
one or more sensors for measuring a positions of a corresponding strut; and
a controller for producing length information of the one or more struts, based on the positions of the one or more struts measured from the one or more sensors; and
a user terminal interlinked with the wireless fracture reduction robot device and configured to receive the length information of the one or more struts, receive control information for adjustment of the distance of the one or more struts from a user, and transmit the received control information to the wireless fracture reduction robot device.
